(19) United States
(12) Patent Application Publication
(10) Pub. No.: US 2022/0142598 A1
(43) Pub. Date: May 12, 2022

(54) SUPPRESSION OF INTERFERENCE EFFECTS IN THE CAPACITIVE MEASUREMENT OF BIOELECTRIC SIGNALS

(71) Applicant: Siemens Healthcare GmbH, Erlangen (DE)

(72) Inventor: Ulrich BATZER, Spardorf (DE)

(73) Assignee: Siemens Healthcare GmbH, Erlangen (DE)

(21) Appl. No.: 17/517,088

(22) Filed: Nov. 2, 2021

(30) Foreign Application Priority Data
Nov. 11, 2020 (DE) 10 2020 214 183.5

(51) Int. Cl. A61B 6/00

(52) U.S. Cl. CPC A61B 6/541; A61B 2562/046; A61B 6/4035

(57) ABSTRACT

An interference signal compensation facility in a differential voltage measuring system including a signal measuring circuit for measuring bioelectric signals with a number of useful signal paths, each with a capacitive sensor electrode for the acquisition of a measurement signal, is described. The interference signal compensation facility includes at least one capacitive reference electrode, set up to acquire a reference signal which possibly includes an interference signal generated by an external interference source. Furthermore, the interference signal compensation facility includes an echo compensation unit, set up to filter the measurement signal based upon the capacitively acquired reference signal and to determine an interference-compensated measurement signal. A differential voltage measuring system is also described. Moreover, an X-ray imaging system is described. In addition, a method for generating an interference-reduced biological measurement signal is described.

### PRIORITY STATEMENT

The present application hereby claims priority under 35 U.S.C. § 119 to German patent application number DE 102020214183.5 filed Nov. 11, 2020, the entire contents of which are hereby incorporated herein by reference.

### FIELD

Example embodiments of the invention generally relate to an interference signal compensation facility. The interference signal compensation facility is set up in a differential voltage measuring system embodied with a signal measuring circuit for measuring bioelectric signals. To measure the bioelectric signals, the differential voltage measuring system comprises a number of useful signal paths each with a capacitive measuring electrode for the acquisition of a measurement signal. Example embodiments of the invention additionally relates to a differential voltage measuring system. Moreover, example embodiments of the invention relates to an X-ray imaging system. In addition, example embodiments of the invention relates to a method for generating an interference-reduced biological measurement signal.

### BACKGROUND

Voltage measuring systems, in particular differential voltage measuring systems, for measuring bioelectric signals are, for example, used in medicine for measuring electrocardiograms (ECGs), electroencephalograms (EEGs) or electromyograms (EMGs).

Usually, the aforementioned measurements are performed using electrodes, which are fastened to a patient's body. As an alternative approach, for some time investigations have been performed with capacitive ECG measurements in which an ECG signal is acquired purely capacitively without direct contact between the capacitive sensors and the patient. In this way, it is, for example, possible to perform an ECG measurement on a clothed patient.

However, as with the conventional measurement of bioelectric signals with electrodes, interference effects also occur with a purely capacitive measurement. One example of such interference effects is ECG signal interference due to X-rays. ECG measurements are often performed during X-ray imaging, for example in order to coordinate the imaging suitably with the heart rate.

One option for suppressing interference due to X-rays is to use a larger number of sensors of which, controlled by the system, preference is given in each case to the sensors which are not currently in the beam path and are therefore also not affected by the interference signals due to X-rays.

### SUMMARY

The inventors have discovered that a disadvantage of the above method is that two complete sensors, including possibly complex mechanical bearings, are required for each contact position on the body.

At least one embodiment of the present invention enables a differential capacitive measurement of bioelectric signals with a simpler measuring arrangement, wherein interference in the measurement, in particular due to X-rays, is suppressed or at least reduced.

Embodiments of the present invention are directed to an interference signal compensation facility; a differential voltage measuring system; an X-ray imaging system; and a method for generating an interference-reduced biological measurement signal.

The interference signal compensation facility according to at least one embodiment of the invention is set up in a differential voltage measuring system with a signal measuring circuit for measuring bioelectric signals with a number of useful signal paths each with a capacitive measuring electrode for the acquisition of a measurement signal and has at least one capacitive reference electrode, which is set up to acquire an interference signal generated by an external interference source, preferably by X-rays, that acts on at least one of the respective capacitive measuring electrodes as a reference signal or reference common-mode interference signal. To acquire the interference signal, the capacitive reference electrode must be located in the area of influence of the same interference source that also triggers an interference signal on the capacitive measuring electrode in question in order to determine this interference signal based upon the measurement by the capacitive reference electrode and to correct the measurement signal of the capacitive measuring electrode in dependence on the interference signal determined.

The differential voltage measuring system according to at least one embodiment of the invention has at least one first capacitive sensor electrode and one second capacitive sensor electrode for measuring bioelectric measurement signals. Furthermore, the differential voltage measuring system according to at least one embodiment of the invention preferably has at least one third capacitive sensor electrode, which is preferably embodied as a reference electrode. The third capacitive sensor electrode can be used to achieve potential equalization between a measurement object and the differential voltage measuring system. This third capacitive sensor electrode can then be used to generate a reference common-mode interference signal that can also be used to correct and filter the measurement signal acquired by the other two sensor electrodes. Moreover, the differential voltage measuring system according to the invention has a measuring facility. The measuring facility has a signal measuring circuit for measuring the bioelectric signals. Furthermore, the measuring facility also has a reference signal unit which generates the aforementioned reference common-mode interference signal and for this purpose is connected to both the aforementioned third capacitive sensor electrode and the signal measuring circuit.

The X-ray imaging system according to at least one embodiment of the invention, preferably a computed tomography system, has an X-ray imaging unit for recording images of an examination region of an examination object. Furthermore, the X-ray imaging system according to at least one embodiment of the invention comprises a differential voltage measuring system according to at least one embodiment of the invention set up to measure a capacitive measurement signal from an examination object, for example an ECG measurement signal. Finally, the X-ray imaging system according to at least one embodiment of the invention comprises a control unit for actuating the X-ray imaging unit in dependence on the capacitive measurement signal acquired from the examination object by the differential voltage measuring system.

The method according to at least one embodiment of the invention for generating an interference-reduced biological measurement signal takes place in a differential voltage measuring system with a signal measuring circuit for measuring bioelectric signals with a number of useful signal paths each with a capacitive sensor electrode for the acquisition of a measurement signal.

In this respect, at least one embodiment of the invention is directed to a corresponding computer program product with a computer program, which can be loaded directly into a memory facility of a capacitive differential voltage measuring system, with program sections for executing all the steps of the method according to at least one embodiment of the invention when the program is executed in the differential voltage measuring system. In addition to the computer program, such a computer program product can optionally include additional parts such as, for example, documentation and/or additional components, and also hardware components, such as, for example, hardware keys (dongles etc.) for using the software.

At least one embodiment of the invention is directed to an interference signal compensation facility in a differential voltage measuring system including a signal measuring circuit for measuring bioelectric signals including a number of useful signal paths, each signal path of the number of useful signal paths including a capacitive sensor electrode for acquisition of a measurement signal, the interference signal compensation facility comprising:

at least one capacitive reference electrode, set up to acquire a reference signal; and

an echo compensation unit, set up to filter the measurement signal based upon the reference signal capacitively acquired and to determine an interference-compensated measurement signal.

At least one embodiment of the invention is directed to a differential voltage measuring system, comprising:

at least one first capacitive electrode and one second capacitive electrode to measure bioelectric measurement signals; and

a measuring facility including
a signal measuring circuit to measure the bioelectric measurement signals, and
the interference signal compensation facility of an embodiment.

At least one embodiment of the invention is directed to an X-ray imaging system, comprising:

an X-ray imaging unit to record images of an examination region of an examination object;

the differential voltage measuring system of an embodiment, set up to measure a capacitive measurement signal on an examination object; and

a control unit to actuate the X-ray imaging unit in dependence on the capacitive measurement signal acquired from the examination object by the differential voltage measuring system.

At least one embodiment of the invention is directed to a method for generating an interference-reduced biological measurement signal in a differential voltage measuring system with a signal measuring circuit for measuring bioelectric signals including a number of useful signal paths, each signal path of the number of signal paths including a capacitive sensor electrode for acquisition of a measurement signal, the method comprising:
capacitive acquisition of a potentially interference-afflicted measurement signal;
capacitive acquisition of a reference signal, potentially including an interference signal generated by an external interference source; and
determining an interference-reduced measurement signal by adaptive filtering of the potentially interference-afflicted measurement signal based upon the reference signal capacitively acquired.

At least one embodiment of the invention is directed to a non-transitory computer program product storing a computer program, directly loadable into a memory facility of a voltage measuring system, including program sections for executing the method of an embodiment when the computer program is executed in the voltage measuring system.

At least one embodiment of the invention is directed to a non-transitory computer-readable medium storing program sections, readable and executable by a computer unit, to execute the method of an embodiment when the program sections are executed by the computer unit.

### BRIEF DESCRIPTION OF THE DRAWINGS

The invention is explained again below in more detail with reference to the attached figures and with reference to example embodiments. Herein, the same components are given identical reference characters in the different figures.

The figures are not generally to scale. In the figures:

FIG. 1 schematically shows a capacitive differential voltage measuring system including possible positioning of the capacitive sensors on a patient,

FIG. 2 shows a schematic depiction of a measuring set-up for the compensated differential measurement of capacitive signals,

FIG. 3 shows a schematic depiction of an X-ray interference signal compensation facility according to an example embodiment of the invention,

FIG. 4 shows a flow diagram schematically depicting a method for generating an interference-reduced biological measurement signal,

FIG. 5 shows a schematic depiction of a computed tomography system according to an example embodiment of the invention.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The interference signal compensation facility according to at least one embodiment of the invention is set up in a differential voltage measuring system with a signal measuring circuit for measuring bioelectric signals with a number of useful signal paths each with a capacitive measuring electrode for the acquisition of a measurement signal and has at least one capacitive reference electrode, which is set up to acquire an interference signal generated by an external interference source, preferably by X-rays, that acts on at least one of the respective capacitive measuring electrodes as a reference signal or reference common-mode interference signal. To acquire the interference signal, the capacitive reference electrode must be located in the area of influence of the same interference source that also triggers an interference signal on the capacitive measuring electrode in question in order to determine this interference signal based upon the measurement by the capacitive reference electrode and to correct the measurement signal of the capacitive measuring electrode in dependence on the interference signal determined.

Capacitive electrodes should be understood to be electrodes which are galvanically separated from the patient or electrically connected to the patient via a high impedance of more than 1 MOhm. This is also intended to include capacitive sensors that are separated from a patient's body by clothing or another layer that is more or less electrically insulating or has poor electrical conductivity.

It is even advantageous for the uppermost layer of a capacitive sensor to have conductive properties, hence creating a weak galvanic connection to a patient or an examination object. One advantage of a weak galvanic connection is that it enables better signal transmission for signal components with low frequencies. If an ohmic resistor is connected in parallel to a capacitor, this arrangement forms a lower impedance for the low frequencies than the capacitor alone. In addition, such a connection enables discharge in the context of ESD protection. Furthermore, in the event that a maximal ohmic resistance is specified, for example 100 MOhm, the requirements for the input impedance of the measuring circuit connected to the sensor electrode or its input circuit are reduced since the ohmic resistance of the sensor electrode forms a voltage divider with the input impedance. This is the case when the patient's clothing is sufficiently electrically conductive, in particular not made of wool.

In this context, a measuring sensor or measuring electrode is intended to refer to a capacitive sensor electrode with which the actual measurement signal on a patient or an examination object, for example a person or an animal, is, as described above, substantially acquired capacitively.

The interference signal compensation facility according to at least one embodiment of the invention also comprises an echo compensation unit, which is set up to filter the useful signal or measurement signal based upon the reference signal and to determine an interference-compensated useful signal or measurement signal. The capacitive reference electrode is arranged such that it does not acquire the capacitive measurement signal. Hence, the capacitive reference electrode acquires a reference signal which can be used as the basis for a filtering process for the measurement signal without the useful component of the measurement signal being lost in the process.

For example, the capacitive reference electrode is arranged congruently behind the capacitive measuring sensor or the capacitive measuring electrode such that the reference electrode is covered by the assigned capacitive measuring electrode and only acquires a ground potential of the signal measuring circuit and not the measurement signal. For this purpose, an intermediate layer or interlayer, which is electrically connected to the ground potential of the signal measuring circuit, can be arranged between the measuring electrode or sensor electrode and the reference electrode, so that the reference electrode is shielded or even electrically insulated from the measurement signal by this intermediate layer or interlayer.

The advantage of the interference signal compensation facility according to at least one embodiment of the invention over a conventional arrangement with a large number of optionally connectable capacitive measuring sensors arranged at different positions is that the possibly very complex mechanical substructure of such a sensor is dispensed with. The reference sensor can preferably be simply deposited in the measuring sensor. For small patients, for whom a plurality of sensors in series are problematic with regard to good contact with the body, this problem is solved by using only one sensor electrode or two parallel sensors connected in parallel on the patient's body. Compared to purely software-based interference correction, the reference signal measurement offers the possibility of also acquiring random effects that are otherwise difficult to model. The interference compensation also has a real-time capability since the adaptive filter only generates a short delay on the reference signal. This is because, during the course of the filtering, the measurement signal is preferably only modified by subtraction and, as a result, apart from the short computing time required, does not experience any delay due to signal processing. In addition, it is also possible to compensate interference in the middle of the frequency band used, for example of an ECG from 0.5 Hz to 40 Hz, and which cannot, therefore, be suppressed by a purely frequency-selective filter, for example a bandpass filter or lowpass filter.

If the capacitive measurement signal, for example an ECG signal, is used in the context of the actuation of an X-ray imaging facility, for example a computed tomography facility, to adapt the imaging process to a dynamic physiological process of an examination object, such as, for example, a patient's heart movement in order to improve image quality, the interference-reduced capacitive measurement signal can be used to adapt the process of X-ray image recording more precisely to the dynamic physiological states represented by the capacitive measurement signal due to the improved quality of the capacitive measurement signal, so that the image quality of the X-ray image recording is further improved.

As mentioned in the introduction, the differential capacitive voltage measuring system acquires bioelectric signals for example from a human or animal patient. For this purpose, it has a number of measuring leads or useful signal paths. These connect, for example as individual cables, the capacitive sensors, attached to the patient for the acquisition of the signals, to the other components of the voltage measuring system, i.e., in particular the electronics used to evaluate or depict the signals acquired.

The basic mode of operation of differential voltage measuring systems is known to the person skilled in the art and so no more detailed explanation will be given here. They can in particular be embodied as electrocardiograms (ECGs), electroencephalograms (EEGs) or electromyograms (EMGs).

The differential voltage measuring system according to at least one embodiment of the invention has at least one first capacitive sensor electrode and one second capacitive sensor electrode for measuring bioelectric measurement signals. Furthermore, the differential voltage measuring system according to at least one embodiment of the invention preferably has at least one third capacitive sensor electrode, which is preferably embodied as a reference electrode. The third capacitive sensor electrode can be used to achieve potential equalization between a measurement object and the differential voltage measuring system. This third capacitive sensor electrode can then be used to generate a reference common-mode interference signal that can also be used to correct and filter the measurement signal acquired by the other two sensor electrodes. Moreover, the differential voltage measuring system according to at least one embodiment of the invention has a measuring facility. The measuring facility has a signal measuring circuit for measuring the bioelectric signals. Furthermore, the measuring facility also has a reference signal unit which generates the aforementioned reference common-mode interference signal and for this purpose is connected to both the aforementioned third capacitive sensor electrode and the signal measuring circuit.

Thus, to measure the reference common-mode interference signal, the differential voltage measuring system preferably has a third useful signal path with the aforementioned third capacitive sensor. Furthermore, the differential voltage measuring system preferably comprises a driver circuit connected between a current measuring resistor and the signal measuring circuit. The driver circuit is also called a “right-leg drive” (RLD) and is responsible for generating a signal regulated to the mean common-mode voltage of individual or all measurement signals. This enables the aforementioned and measured common-mode interference signals in the useful signal paths to be eliminated.

The third useful signal path (or “right-leg drive path”) provides potential equalization between the patient and the capacitive differential voltage measuring system or the ECG measuring system. Herein, the capacitive sensor of the third useful signal path is preferably attached to the patient's right leg, to which the term “right-leg drive” is attributable. However, in principle, this third potential can also be acquired elsewhere on the patient.

In addition, the differential voltage measuring system according to at least one embodiment of the invention also has the interference signal compensation facility according to at least one embodiment of the invention. The differential voltage measuring system according to at least one embodiment of the invention shares the advantages of the interference signal compensation facility according to at least one embodiment of the invention.

The X-ray imaging system according to at least one embodiment of the invention, preferably a computed tomography system, has an X-ray imaging unit for recording images of an examination region of an examination object. Furthermore, the X-ray imaging system according to at least one embodiment of the invention comprises a differential voltage measuring system according to at least one embodiment of the invention set up to measure a capacitive measurement signal from an examination object, for example an ECG measurement signal. Finally, the X-ray imaging system according to at least one embodiment of the invention comprises a control unit for actuating the X-ray imaging unit in dependence on the capacitive measurement signal acquired from the examination object by the differential voltage measuring system.

Advantageously, the interference-reduced capacitive measurement signal can be used to adapt the X-ray image recording process to dynamic physiological states represented by the capacitive measurement signal, such as, for example, a patient's heart movement so that the image quality of the X-ray image recording is improved. Such an adaptation can, for example, take place by synchronization of image recording time intervals and/or rotation of a movable image recording unit, such as, for example, a rotating detector or a rotating scanner unit, with these physiological states or dynamic processes.

The method according to at least one embodiment of the invention for generating an interference-reduced biological measurement signal takes place in a differential voltage measuring system with a signal measuring circuit for measuring bioelectric signals with a number of useful signal paths each with a capacitive sensor electrode for the acquisition of a measurement signal.

In the method according to at least one embodiment of the invention, a possibly interference-afflicted measurement signal is acquired capacitively. In addition, a reference signal, which may possibly be affected by an interference signal generated by an external interference source, is acquired capacitively.

The acquisition of the reference signal preferably takes place galvanically separated from the acquisition of the measurement signal or with a high impedance of preferably more than 1 MOhm in relation to the measuring path of the measurement signal, so that the reference signal is not influenced, or is influenced as little as possible, by the measurement signal. Furthermore, the acquisition of the reference signal preferably takes place spatially associated with the acquisition of the measurement signal, so that the interference effects that influence the measurement signal sufficiently match the interference effects that generate the reference interference signal.

Finally, an interference-reduced measurement signal is generated by adaptive filtering of the possibly interference-afflicted measurement signal in dependence on the reference signal. In other words, the reference signal is used to determine the interference effects on the measurement signal and a possibly dynamic transfer function between the measuring path and the reference signal path is determined or continuously adapted with the adaptive filter. The method according to at least one embodiment of the invention for generating an interference-reduced biological measurement signal shares the advantages of the interference signal compensation facility according to at least one embodiment of the invention.

A large part of the aforementioned components of the interference signal compensation facility according to at least one embodiment of the invention, in particular the echo compensation unit with an adaptive filter function, can be wholly or partially implemented in the form of software modules in a processor of a corresponding capacitive differential voltage measuring system, wherein additional hardware elements, such as, for example, a capacitive reference electrode, preferably in the immediate vicinity of the associated measuring electrode, and/or extended front-end hardware or electronic circuitry of the capacitive reference electrode, which is to be supplemented accordingly, are to be added. An extensively software-based implementation has the advantage that it is also possible to retrofit capacitive differential voltage measuring systems used to date in a simple way via a software update in order to work in the manner according to at least one embodiment of the invention.

In this respect, at least one embodiment of the invention is directed to a corresponding computer program product with a computer program, which can be loaded directly into a memory facility of a capacitive differential voltage measuring system, with program sections for executing all the steps of the method according to at least one embodiment of the invention when the program is executed in the differential voltage measuring system. In addition to the computer program, such a computer program product can optionally include additional parts such as, for example, documentation and/or additional components, and also hardware components, such as, for example, hardware keys (dongles etc.) for using the software.

Transportation to the differential voltage measuring system and/or storage on or in the differential voltage measuring system can take place via a computer-readable medium, for example a memory stick, a hard disk or another kind of transportable or integrated data carrier on which the program sections of the computer program which can be read and executed by a computer unit of the differential voltage measuring system are stored. For this purpose, the computer unit can, for example, have one or more interacting microprocessors or the like.

Further particularly advantageous embodiments and developments of the invention emerge from the dependent claims and the following description, wherein the claims of one category of claims can also be developed analogously to the claims and descriptive passages to create another category of claims and in particular individual features of different example embodiments or variants can be combined to create new example embodiments or variants.

The interference signal compensation facility according to at least one embodiment of the invention is preferably embodied such that in each case a, preferably in each case one single, capacitive reference electrode is assigned to a capacitive sensor electrode in each case. Herein, particularly preferably, a respective capacitive reference electrode is in each case arranged spatially associated with a respective capacitive sensor electrode, so that external interference acts approximately equally on the capacitive reference electrode and the capacitive sensor electrode assigned thereto.

“Spatially associated” is intended to mean that the reference electrode is spatially positioned sufficiently close to the associated sensor electrode to ensure that an X-ray spectrum that acts on the sensor electrode deviates only insignificantly in terms of energy distribution and intensity from the X-ray spectrum that acts on the reference electrode. In this context, “insignificantly” in this context is intended to mean that the error in the interference signal compensation does not exceed a predetermined extent. The smaller the extent of the deviation, the fewer iterations an estimation filter has to perform in order to compensate the error caused by the deviation. This advantageously reduces the computational effort in the filtering process of the echo compensation unit and hence improves the real-time capability of the system.

It is quite particularly preferable for the capacitive sensor electrode and the capacitive reference electrode to be arranged one behind the other and preferably parallel to one another. Such an arrangement makes it possible that interference acting approximately perpendicularly on the capacitive sensor electrode, for example X-rays, also acts in the same way on the capacitive reference electrode insofar as the interference is transmitted by the capacitive sensor electrode, which is the case with X-rays. Due to the approximate equality of the interference effects on the two sensors, the reference signal of the capacitive reference electrode can map the interference acting on the capacitive sensor electrode particularly exactly. Advantageously, this improves the quality of the filtering process and possibly also reduces the computational effort in the filtering process of the echo compensation unit and consequently improves the real-time capability of the overall system.

It is also very preferable for the capacitive sensor electrode and the capacitive reference electrode to be arranged one behind the other at a short distance, preferably at a distance of a few centimeters, even more preferably at a distance of a few millimeters, even more preferably at a distance less than one millimeter, to ensure that, regardless of the direction from which it is incident or acts on the capacitive sensor electrode, interference acting on the capacitive sensor electrode also acts in the same way on the capacitive reference electrode. Due to the equality of the interference effects on the two sensors, the reference signal can map the interference acting on the capacitive sensor electrode particularly exactly. Advantageously, this improves the quality of the filtering process and possibly also reduces the computational effort in the filtering process of the echo compensation unit and consequently also improves the real-time capability of the overall system.

It is also very preferable for the capacitive reference electrode to be arranged congruently with a capacitive sensor electrode spatially associated therewith. In this case, “congruently” is intended to mean that the two sensor surfaces are not only arranged one behind the other and parallel, preferably at a short distance from one another but also completely cover one another when viewed from a direction oriented perpendicular to their sensor surface.

Advantageously, such an arrangement achieves a particularly exact identity or homogeneity of the interference acting on the two sensors. Due to the equality of the interference effects on the two sensors, the reference signal can map the interference acting on the capacitive sensor electrode particularly exactly. Advantageously, this improves the quality of the filtering process and possibly also reduces the computational effort in the filtering process of the echo compensation unit and consequently also improves the real-time capability of the overall system still further.

The interference signal compensation facility according to at least one embodiment of the invention is preferably embodied such that a respective capacitive reference electrode is galvanically separated from the respective measuring electrode or at least electrically connected to the respective measuring electrode with a high impedance of preferably more than 1 MOhm. Herein, however, the respective capacitive reference electrode is preferably spatially associated with the respective measuring electrode, particularly preferably congruently with the respective measuring electrode, and set up to acquire a reference signal or reference common-mode interference signal. The insulation of the two capacitive electrodes ensures that, in contrast to the interference, the measurement signal does not influence the reference signal so that the measurement information or the desired component of the measurement signal is not unintentionally impaired by the subsequent filtering based upon the reference signal.

The echo compensation unit of the interference signal compensation facility according to at least one embodiment of the invention is preferably embodied such that a filter function is adapted to a transfer function between the reference electrode and the sensor electrode based upon a mixed signal. To estimate the transfer function, the echo compensation unit preferably has an adaptive filter unit which can be adapted to dynamic interference behavior. To generate the mixed signal, the echo compensation unit preferably comprises a mixing unit. The mixed signal generated by the mixing unit includes a reference interference signal modified by an estimated transfer function and a measurement signal mixed therewith. The mixing of the two signals can, for example, be implemented by subtracting the reference interference signal modified by an estimated transfer function from the measurement signal. The estimated signal generated in this way is then used again by the adaptive filter unit to generate a corrected transfer function, as a result of which an interference-reduced measurement signal is generated iteratively. Advantageously, the iterative process achieves a high degree of accuracy in the estimation process of the measurement signal and a high degree of efficiency of the filtering process.

The interference signal compensation facility preferably also comprises a so-called front-end hardware unit set up to preprocess the sensor signal and the reference interference signal. Typical preprocessing steps include buffering, amplification and digitization of the measurement signal and the reference interference signal.

The echo compensation unit of the interference signal compensation facility according to at least one embodiment of the invention is particularly preferably set up to determine an optimized transfer function based upon a least mean square method. Alternatively, the echo compensation unit can be set up to determine an optimized transfer function, based upon a recursive least square method. Particularly preferably, the aforementioned optimization method generates a minimized measurement signal that includes minimal interference components. Advantageously, an interference-reduced measurement signal can be generated even if the transfer function between the reference signal and the interference signal actually present on the measurement signal changes dynamically.

In each of the figures, it is assumed by way of example that an ECG differential voltage measuring system 1 is the differential voltage measuring system 1 for measuring bioelectric signals S(k), here ECG signals S(k). However, the invention is not restricted thereto.

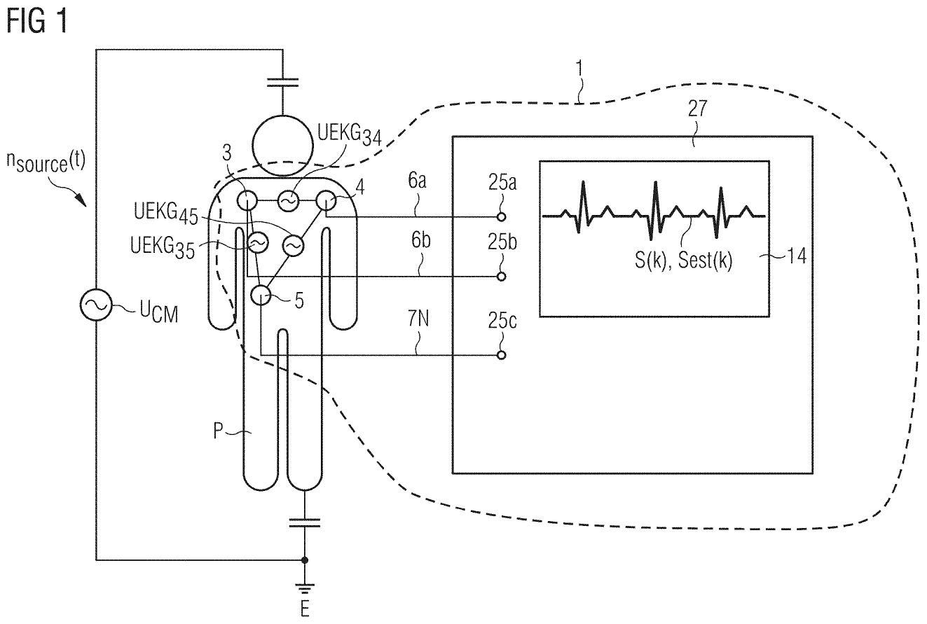

FIG. 1 shows by way of example an ECG measuring system 1 according to an embodiment of the invention, namely a schematic depiction of an ECG device 27 with its electrical connectors and capacitive electrodes 3, 4, 5, connected thereto via cables K in order to measure ECG signals S(k) on a patient P. This ECG measuring system 1 is able, with the aid of the invention, to suppress interference signals coupled into the electrodes 3, 4, 5. Capacitive electrodes should be understood to be electrodes that are galvanically separated from the patient or electrically connected to the patient via a high impedance.

In order to measure the ECG signals S(k), at least one first capacitive sensor electrode 3, also referred to as the first capacitive sensor, and a second capacitive sensor electrode 4, also referred to as the second capacitive sensor, are required; these are attached to the patient P, but galvanically separated from the patient P or electrically connected to the patient P via a high impedance. The capacitive sensors 3, 4 are connected to the ECG device 27 by means of signal measuring cables K via connectors 25a, 25b, generally plug-in connections 25a, 25b. Herein, the first capacitive sensor 3 and the second capacitive sensor 4 including the signal measuring cables 6a, 6b form part of a signal acquisition unit with which the ECG signals S(k) can be acquired.

A third capacitive sensor 5 serves as a compensation sensor to create potential equalization between the patient P and the ECG device 27. This compensation sensor 5 will be explained in more detail later. This third electrode 5 is conventionally attached to the patient's right leg (which is why, as mentioned above, this connector is often referred to as a “right-leg drive” or “RLD”). However, as is also the case here, it can be positioned elsewhere. In addition, a large number of further contacts for further leads (potential measurements) can be attached to the patient P via further connectors, not depicted in the figures, on the ECG device 27 and used to form suitable signals.

The voltage potentials UEKG₃₄, UEKG₄₅ and UEKG₃₅, which serve to measure the ECG signals S(k), are formed between the individual capacitive sensors 3, 4, 5.

The directly measured ECG signals S(k) and/or further-processed bioelectric signals Sₑₛₜ(k) are displayed on a user interface 14 of the ECG device 27.

During the ECG measurement, the patient P is at least capacitively coupled to the ground potential E (schematically depicted in FIG. 1 by a coupling on the head and the right leg). However, it is subject to an interference source U_{CM} and the interference signal n_{source}(t) resulting therefrom which is present across the patient P and changes constantly with time t, which is inevitably also acquired by the relatively sensitive measurement. This interference source U_{cm} generally couples interference signals via the patient P into the sensors 3, 4; this will be referred to later. In addition, interference signals can also be generated by direct action on the sensor electrodes 3, 4, 5. This is, for example, the case when X-rays act on the sensors 3, 4, 5.

Herein, the signal measuring cables leading from the first electrode 3 and the second electrode 4 to the ECG device 27 are part of the useful signal paths 6a, 6b. Herein, the signal measuring cable leading from the electrode 5 to the ECG device 27 corresponds to part of a third useful signal path 7N. The third useful signal path 7N transmits interference signals from the interference source Ucm, which were coupled in via the patient P and the electrodes.

As already mentioned, in addition to the above-described interference source Ucm, which can, for example, be a 60 Hz voltage source, X-rays also occur as causes of interference during ECG monitoring of a CT recording and these also influence the measurement signal S(t).

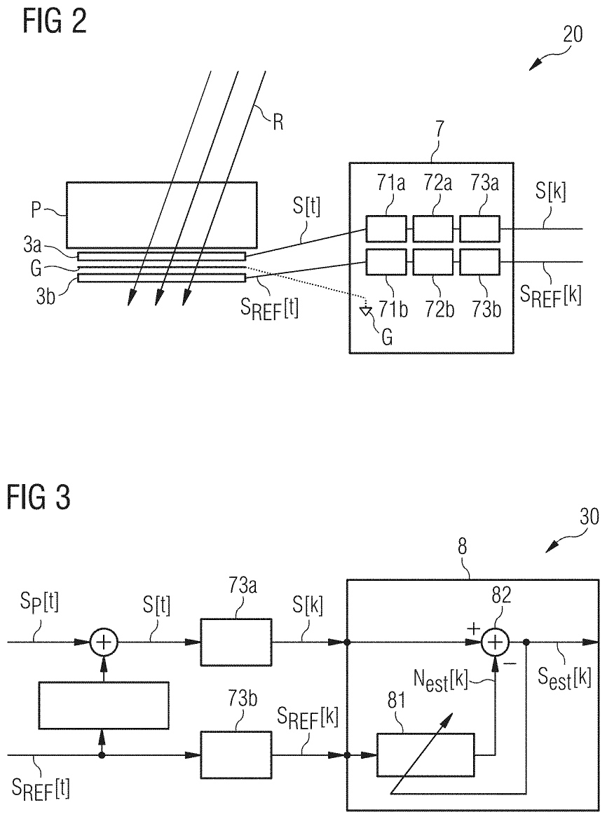

FIG. 2 is a schematic depiction 20 of a measuring arrangement for compensated differential measurement of capacitive signals. The arrangement comprises a capacitive sensor 3a positioned on a patient P and galvanically separated therefrom, which is set up to acquire a measurement signal S(t) from the patient. A capacitive reference sensor 3b, which is electrically insulated from the capacitive sensor 3a and capacitively acquires the ground potential G instead of the patient signal, is arranged behind the capacitive sensor 3a. Since the capacitive reference sensor 3b is arranged congruently behind the capacitive sensor 3a, it is penetrated by the same X-rays R as the capacitive sensor 3a.

Consequently, the capacitive reference sensor 3b can generate a reference signal S_{REF}(t) afflicted by the same interference. However, the measurement signal S(t) does not simply include the reference signal S_{REF}(t), but rather an interference signal corresponding to a reference signal S_{REF}(t) transformed by a transfer function h(t). Both signals S(t), S_{REF}(t) are transmitted to a so-called front-end unit 7, which in each case comprises a signal buffer 71a, 71b for the respective signals S(t), S_{REF}(t), a respective amplifier 72a, 72b and a respective AD converter 73a, 73b.

FIG. 3 is a schematic depiction of an interference compensation circuit 30 according to an example embodiment of the invention. The measurement process is shown schematically on the left of the figure. During the measurement process, the pure measurement signal S_{P}(t) becomes an interference-afflicted measurement signal S(t) due to interference corresponding to the transfer of the reference signal S_{REF}(t) by a transfer function h(t) to the non-interference afflicted measurement signal S_{P}(t). In FIG. 3, processing by the front-end hardware is only symbolized by the AD converters 73a, 73b. The digitized signals S(k), S_{REF}(k) are transmitted to an echo compensation unit 8.

The echo compensation unit 8 comprises an adaptive filter unit 81 and a mixing unit 82. The echo compensation unit 8 is set up to generate a first interference-reduced measurement signal based upon the measurement signal S(k) and the reference signal S_{REF}(k). The adaptive filter unit 81 is set up to estimate a transfer function h(t). The reference signal Nₑₛₜ(k) modified by the transfer function h(t) is then subtracted from the measurement signal S(k) in the mixing unit 81 in order to reduce the interference components of the measurement signal S(k) caused by the X-rays. Furthermore, the echo compensation unit 8 has the property of being able to change its transfer function h(t) and its frequency independently during operation. Herein, an error signal, for example the actual estimated measurement signal Sₑₛₜ(k), is generated in dependence on an output signal Sₑₛₜ(k) from the mixing unit 81 of the echo compensation unit 8, and the filter coefficients h_{f} of the transfer function h(t) estimated by the adaptive filter unit 81 are changed to estimate the transfer function h(t) in dependence on the error signal Sₑₛₜ(k) such that the error signal Sₑₛₜ(k) is minimized. The echo compensation unit 8, possibly after a plurality of iteration loops, finally outputs a compensated measurement signal Sₑₛₜ(k) which is free of the interference effects caused by the X-rays R.

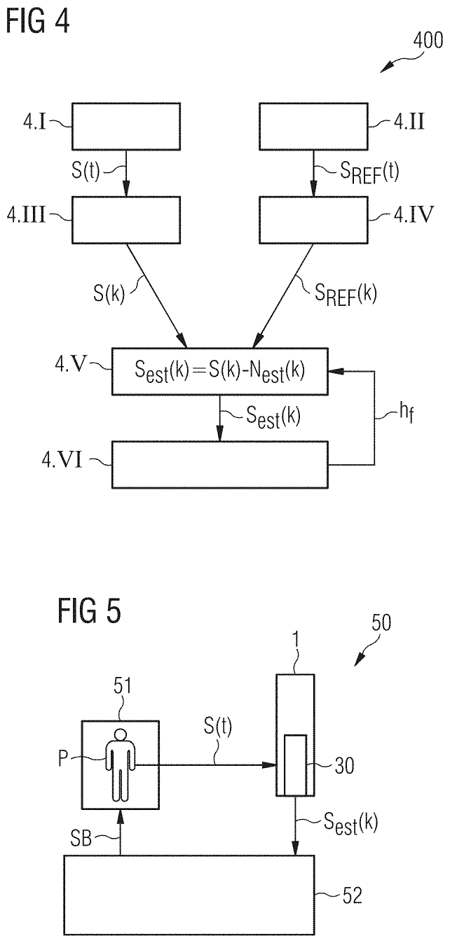

FIG. 4 is a flow diagram 400 schematically illustrating a method for generating an interference-reduced biological measurement signal Sₑₛₜ(t). In step 4.I of the method, a possibly interference-afflicted measurement signal S(t) is acquired on a patient who is currently lying in an X-ray imaging facility and exposed to X-rays. The X-rays are incident on the capacitive sensors 3, 4 of a capacitive differential voltage measuring system and generate the interference-afflicted signal S(t). In step 4.II, a capacitive reference interference signal S_{REF}(t) is acquired by a reference sensor 3b. In step 4.III, the measurement signal S(t) is preprocessed; this includes buffering, amplifying and generating a digital measurement signal S(k). Simultaneously, in step 4.IV, the reference signal S_{REF}(t) acquired in step 4.II is preprocessed, wherein a digitized reference signal S_{REF}(k) is generated. In step 4.V, the digital measurement signal S(k) is then filtered with the digital reference signal S_{REF}(k).

In step 4.V, the digital reference signal S_{REF}(k) is fed to an adaptive filter unit 81 (see FIG. 3) of an echo compensation unit 8 (see FIG. 3 in each case), which first estimates a transfer function h(t) with so-called filter coefficients h_{f}. The estimated transfer function h(t) can now be used to determine an estimated interference signal Nₑₛₜ(k) based upon the reference signal S_{REF}(k). The estimated interference signal Nₑₛₜ(k) is now subtracted from the measurement signal S(k) and the filter coefficients h_{f} of the estimation unit are adapted in step 4.VI in dependence on the result of the estimation Sₑₛₜ(k). In the context of an iteration, a return is made to step 4.V and a new estimation of the transfer function h(t) is performed with the new filter coefficients h_{f}. The new transfer function h(t) is used as the basis for a new estimation of an interference signal Nₑₛₜ(k) which is subtracted from the measurement signal S(k) and an estimated result Sₑₛₜ(k) is generated. The iteration between step 4.V and 4.VI continues until the estimated measurement signal Sₑₛₜ(k) is optimized. This state is, for example, reached when a change in the estimated measurement signal Sₑₛₜ(k) has fallen below a predetermined threshold during a run through an iteration loop.

FIG. 5 is a schematic depiction of a computed tomography system 50, CT system for short, according to an example embodiment of the invention. The CT system 50 comprises a scanner unit 51 for recording images of an examination region of an examination object or a patient P. Furthermore, the CT system 50 comprises a differential voltage measuring system 1 with the interference signal compensation facility according to the invention 30. The differential voltage measuring system 1 acquires a capacitive measurement signal S(t), in this example embodiment an ECG signal, from the patient P. The acquired measurement signal S(t) is transmitted to a control unit 52. Herein, the control unit 52 is set up to actuate the scanner unit 51 in dependence on the acquired capacitive measurement signal S(t) such that the imaging process of the scanner unit 51 is synchronized with the heartbeat motion of the patient P. For this purpose, the control unit 52 transmits control commands SB to the scanner unit 51.

Finally, reference is made once again to the fact that the apparatuses and methods described above in detail are only example embodiments which can be modified in wide ranges by the person skilled in the art without departing from the scope of the invention. For example, the differential voltage measuring system does not need to be an ECG device, it can also entail other medical devices with which bioelectric signals are acquired, such as, for example, EEGs, EMGs etc. Furthermore, the use of the indefinite article “a” or “an” does not preclude the possibility that the features in question may also be present on a multiple basis. Similarly, the term “unit” does not exclude the possibility that the unit could consist of a plurality of components, which could also be spatially distributed.

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An interference signal compensation facility in a differential voltage measuring system including a signal measuring circuit for measuring bioelectric signals including a number of useful signal paths, each signal path of the number of useful signal paths including a capacitive sensor electrode for acquisition of a measurement signal, the interference signal compensation facility comprising:
at least one capacitive reference electrode, set up to acquire a reference signal; and
an echo compensation unit, set up to filter the measurement signal based upon the reference signal capacitively acquired and to determine an interference-compensated measurement signal.

2. The interference signal compensation facility of claim 1, wherein the at least one capacitive reference electrode is set up to acquire an interference signal generated by X-rays as the reference signal.

3. The interference signal compensation facility of claim 1, wherein a respective capacitive reference electrode, of the at least one capacitive reference electrode, is arranged spatially associated with a respective capacitive sensor electrode, so that interference caused by the external interference source acts approximately equally on the respective capacitive reference electrode and a respectively assigned at least one capacitive sensor electrode.

4. The interference signal compensation facility of claim 3, wherein the respective capacitive reference electrode is arranged congruently with a spatially associated capacitive sensor electrode.

5. The interference signal compensation facility of claim 1, wherein the at least one capacitive reference electrode is arranged galvanically separated from the respective capacitive sensor electrode.

6. The interference signal compensation facility of claim 1, wherein the at least one capacitive reference electrode is arranged electrically insulated from the respective capacitive sensor electrode with a minimum impedance of 1 MOhm.

7. The interference signal compensation facility of claim 1, wherein the echo compensation unit is set up to adapt a filter function of the echo compensation unit to a transfer function between the at least one reference electrode and the at least one sensor electrode based upon a mixed signal.

8. The interference signal compensation facility of claim 1, further comprising a front-end hardware unit, set up to buffer, amplify and digitize the measurement signal and the reference signal.

9. The interference signal compensation facility of claim 1, wherein the echo compensation unit is set up to determine an optimized estimated transfer function based upon a least mean square method in order to adapt the filter function.

10. The interference signal compensation facility of claim 1, wherein the echo compensation unit is set up to determine an optimized estimated transfer function based upon a recursive least square method in order to adapt the filter function.

11. A differential voltage measuring system, comprising:
at least one first capacitive electrode and one second capacitive electrode to measure bioelectric measurement signals; and
a measuring facility including
a signal measuring circuit to measure the bioelectric measurement signals, and
the interference signal compensation facility of claim 1.

12. An X-ray imaging system, comprising:
an X-ray imaging unit to record images of an examination region of an examination object;
the differential voltage measuring system of claim 11, set up to measure a capacitive measurement signal on an examination object; and
a control unit to actuate the X-ray imaging unit in dependence on the capacitive measurement signal acquired from the examination object by the differential voltage measuring system.

13. A method for generating an interference-reduced biological measurement signal in a differential voltage measuring system with a signal measuring circuit for measuring bioelectric signals including a number of useful signal paths, each signal path of the number of signal paths including a capacitive sensor electrode for acquisition of a measurement signal, the method comprising:
capacitive acquisition of a potentially interference-afflicted measurement signal;
capacitive acquisition of a reference signal, potentially including an interference signal generated by an external interference source; and
determining an interference-reduced measurement signal by adaptive filtering of the potentially interference-afflicted measurement signal based upon the reference signal capacitively acquired.

14. A non-transitory computer program product storing a computer program, directly loadable into a memory facility of a voltage measuring system, including program sections for executing the method of claim 13 when the computer program is executed in the voltage measuring system.

15. A non-transitory computer-readable medium storing program sections, readable and executable by a computer unit, to execute the method of claim 13 when the program sections are executed by the computer unit.

16. The interference signal compensation facility of claim 1, wherein the at least one capacitive reference electrode is set up to acquire a reference signal including an interference signal generated by an external interference source.

17. The interference signal compensation facility of claim 2, wherein a respective capacitive reference electrode, of the at least one capacitive reference electrode, is arranged spatially associated with a respective capacitive sensor electrode, so that interference caused by the external interference source acts approximately equally on the respective capacitive reference electrode and a respectively assigned at least one capacitive sensor electrode.

18. The interference signal compensation facility of claim 17, wherein the respective capacitive reference electrode is arranged congruently with a spatially associated capacitive sensor electrode.

19. The interference signal compensation facility of claim 2, wherein the at least one capacitive reference electrode is arranged galvanically separated from the respective capacitive sensor electrode.

20. The interference signal compensation facility of claim 2, wherein the at least one capacitive reference electrode is arranged electrically insulated from the respective capacitive sensor electrode with a minimum impedance of 1 MOhm.

21. The interference signal compensation facility of claim 2, further comprising a front-end hardware unit, set up to buffer, amplify and digitize the measurement signal and the reference signal.

22. The interference signal compensation facility of claim 2, wherein the echo compensation unit is set up to determine an optimized estimated transfer function based upon a least mean square method in order to adapt the filter function.

23. The interference signal compensation facility of claim 2, wherein the echo compensation unit is set up to determine an optimized estimated transfer function based upon a recursive least square method in order to adapt the filter function.
